(19) United States
(12) Patent Application Publication
(10) Pub. No.: US 2008/0260857 A1
(43) Pub. Date: Oct. 23, 2008

(54) Substance and Method for Treating Neoplasms

(76) Inventors: Igor Anatolievich Pomytkin, Moscow (RU); Sergey Pavlovich Soloviev, Moscow (RU)

Correspondence Address: NOTARO AND MICHALOS, 100 DUTCH HILL ROAD, SUITE 110, ORANGEBURG, NY, 10962-2100, US

(21) Appl. No.: 12/102,143

(22) Filed: Apr. 14, 2008

Related U.S. Application Data
(63) Continuation of application No. PCT/RU2005/000688, filed on Dec. 30, 2005.

(30) Foreign Application Priority Data
Apr. 1, 2005 (RU) 2005109361

(51) Int. Cl. A61K 33/00; A61P 35/00

(52) U.S. Cl. 424/600

(57) ABSTRACT

The invention relates to substance and method for treating neoplasms in mammals. A substance for treating neoplasm comprises water comprising from 99.76 to 99.99 molecular % of light isotopologue ¹H₂¹⁶O and up to 100% of residual isotopologues. A method for treating neoplasm comprises a step of administering to a mammal in need thereof an effective amount of water comprising from about 99.76 to about 99.99 molecular % of light isotopologue ¹H₂¹⁶O and up to 100% of residual isotopologues. Preferably, the effective amount of the water comprising from about 99.76 to about 99.99 molecular % of light isotopologue ¹H₂¹⁶O and up to 100% of residual isotopologues is administered orally or parenterally. Preferably, the method further comprises a step of administering to a mammal in need thereof from is 0.1 to 50 mg per kg body weight of succinic acid or a pharmaceutically acceptable salt thereof orally or parenterally. The method may further comprise a step of administering to a mammal in need thereof an effective amount of an anti-neoplasm agent. Preferably, the mammal is a human.

### FIELD OF THE INVENTION

The present invention relates to substances and methods for treating neoplasms in mammals, particularly humans.

### BACKGROUND OF THE INVENTION

Neoplasm is any abnormal growth of new tissue which is no longer under normal physiologic control. In accordance with International Statistical Classification of Diseases and Related Health Problems 10th Revision (ICD-10) neoplasms subdivided on malignant (cancerous), benign, in situ neoplasms, and neoplasms of uncertain or unknown behaviour. Malignant neoplasms (cancer) is a group of diseases that are each characterized by abnormal and uncontrolled growth of a particular type of cell known as transformed cells. This deregulated proliferation or an inability of the transformed cells to undergo apoptotic cell, death results in the development of tumors.

Current methods of treating neoplasms include surgery, chemotherapy and radiotherapy. Surgery is typically used as the primary treatment neoplasms, however, many tumors cannot be completely removed by surgical means. In addition, metastatic growth of neoplasms may prevent complete cure of neoplasm by surgery. Radiotherapy cannot be used to treat many neoplasms because of the sensitivity of tissue surrounding the tumor.

Chemotherapy involves administration to mammals in need thereof of chemical compounds having anti-neoplasm activity. The efficacy of chemotherapy is often limited by severe side effects including bone marrow depression, renal damage, central nervous system depression, and nausea and vomiting.

Typically, anti-neoplasm agents inhibit proliferation and induce apoptosis in sensitive tumor cells. Herr and Debatin, Blood, 2001, 98(9): 2603-2614. Frequently, such agents (e.g. paclitaxel, 5-fluorouracil, daunorubicin, and platinum compounds) induce apoptosis in target cells at least in part through the generation of elevated amounts of intracellular reactive oxygen species and hydrogen peroxide (H₂O₂). Varbiro G et al. Free Radic Biol Med 2001; 31:548-58. Hwang P M et al., Nat Med 2001; 7:1111-7. Baker DE. Rev Gastroenterol Disord 2003; 3:31-8. However, the H₂O₂ induces apoptosis in non-transformed as well as transformed cells and, thus, causes the adverse effects such as bone marrow depression. So, there is a need for a safe, effective agent for treating neoplasms that selectively induces apoptosis only in transformed cells in order to avoid side effects like bone marrow depression.

It has been found recently that H₂O₂ differentially affects transformed and non-transformed cells in the narrow range of H₂O₂ concentrations close to highest physiological or slightly supraphysiological range. At concentrations from tens to a hundred nanomoles, H₂O₂ inhibits both proliferation and viability of transformed cells, but, contrary, enhances both proliferation and viability of non-transformed cells. Laurent A et al., Cancer Research 65: 948-956 (2005). So, there is the need in a method that provides the desired H₂O₂ concentrations in cells in order to inhibit selectively both proliferation and viability of transformed cells and simultaneously enhance both proliferation and viability of non-transformed cells. This method could provide desired therapeutic effect in treating neoplasms without side effects like as bone marrow depression.

Mitochondria are major source of H₂O₂ in mammalian body and succinate is the best substrate for H₂O₂ generation by mitochondria. Boveris A et al., Biochem J. 1972, 128: 617-630. Succinate is a human (mammalian) metabolite. Succinate level in human blood is from 1 to about 9 μM while can rise up to 125 μM under hypoxia. Komaromy-Hiller G. et al., Ann. Clin. Lab. Sci. 1997. 27(2): 163-168. Hochachka P et al. Eur. J. Appl. Physiol. 1976. 35(4): 235-242. Succinate dose-dependently supports H₂O₂ generation in mitochondria and provides the rate lower than a nanomole H₂O₂ per a minute at succinate level from 1 to 9 μM. Pomytkin I and Kolesova O. Bull Exp Biol Med. 2003 135(6):541-2. So, endogenous succinate concentrations support H₂O₂ generation in cells at levels insufficient to achieve a significant inhibition of transformed cell proliferation. Thus, there is the need in increasing the rate of succinate-supported H₂O₂ generation in mitochondria in order to provide H₂O₂ concentrations sufficient to achieve the significant anti-neoplasm effect.

It is known from the art that change in the isotopic composition of the solvent results in effect on kinetics or equilibrium of reactions. This effect is lnown as a solvent isotope effect according to IUPAC Compendium of Chemical Terminology 2nd Edition (1997). Typically, the change of a heavy isotope on a light isotope in the isotopic composition of the solvent results in increasing the rate of a reaction. In relation to succinate-supported H₂O₂ generation, light water isotopologue ¹H₂¹⁶O as a solvent may provide a particularly high rate of the H₂O₂ generation as compared to heavy isotopologues (e.g. ¹H₂¹⁸O, ¹H₂¹⁷O, and etc.) and, thus, provide intracellular H₂O₂ concentrations sufficient to achieve the significant anti-neoplasm effect in a mammalian body. The term “isotopologue” refers herein to molecules that differ in isotopic composition, e.g. ¹H₂¹⁶O and ¹H₂¹⁸O.

It is known that natural water represents itself a composition of nine water isotopologues (¹H₂¹⁶O, ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O), wherein the level of light water isotopologue ¹H₂¹⁶O is about 99.7317% (Vienna Standard Mean Ocean Water, VSMOW), and wherein total level of residual eight heavy isotopologues is about 0.2683% (e.g. 0.199983% ¹H₂¹⁸O, 0.0372% ¹H₂¹⁷O, 0.031069% ¹H²H¹⁶O, 0.0000623% ¹H²H¹⁸O, and 0.0000116% ¹H²H¹⁷O). Rothman et al., J. Quant. Spectrosc. Radiat. Transfer, 1998, 60, 665. Rothman et al., J. Quant. Spectrosc. Radiat. Transfer, 2003, 82, p. 9. The abundance of water isotopologues in natural water slightly varies on Earth district and climatic conditions and is expressed typically as the deviation, δ, relative to the international VSMOW standard. The natural water maximally enriched by major light water isotopologue ¹H₂¹⁶O was founded in Antarctica (Standard Light Antarctic Precipitation, SLAP), wherein said 6-values of residual heavy isotopes are δ²H-415.5% ₀, δ¹⁷O-28.1% ₀, and δ¹⁸O-53.9% ₀ that corresponds to the 99.757% level of light water isotopologue ¹H₂¹⁶O. R. van Trigt, Laser Spectrometry for Stable Isotope Analysis of Water Biomedical and Paleoclimatological Applications, 2002, Groningen: University Library Groningen, p. 50. Thus, water with the abundance of light water isotopologue ¹H₂¹⁶O more than 99.757% is not found in nature. Water enriched by light water isotopologue ¹H₂¹⁶O to levels more than 99.76% can be prepared in industrial scale, for example, by highly-effective distillation.

We have found that water enriched by light isotopologue ¹H₂¹⁶O to levels more than 99.76% significantly increases the rate of succinate-supported H₂O₂ generation as compared to usual water with content of light isotopologue ¹H₂¹⁶O about 99.73%. Due to the increase in H₂O₂ generation, it is possible now to achieve H₂O₂ concentrations in cells at levels sufficient for inhibiting both proliferation and viability of transformed cells and simultaneously enhancing both proliferation and viability of non-transformed cells. So, the use of water enriched by light isotopologue ¹H₂¹⁶O provides particularly advantageous methods for treating neoplasms comprising enhancement of succinate-supported H₂O₂ production to desired concentrations. Such methods may comprise administering said water to a mammal in order to enhance H₂O₂ generation supported by endogenous succinate at typical endogenous succinate concentrations are from 1 to 9 μM. Particularly advanced methods for treating neoplasms may further comprise administering said water to a mammal by steps or in a combination with exogenous succinate in order to additively enhance H₂O₂ generation due to elevating succinate concentrations in mammalian body.

It is an object of the present invention to provide a substance for treating neoplasms which comprises water comprising from about 99.76 to about 99.99 molecular % of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O.

It is an object of the present invention to provide a method for treating neoplasms which comprises a step of administering to a mammal in need thereof an effective amount of water comprising from about 99.76 to about 99.99% of light isotopologue ¹H₂¹⁶O and up to 100% of residual isotopologues ¹H₂¹⁷O, ¹H₂¹⁸O, ¹H₂H¹⁶O, ¹H₂H¹⁷O, ¹H²H¹⁸O, ²H₂¹⁷O, ²H₂¹⁸O. Preferably, the method further comprises a step of administering to a mammal in need thereof an effective amount of succinic acid or pharmaceutically acceptable salts thereof, or an anti-neoplasm agent.

### BRIEF DESCRIPTION OF THE DRAWINGS

FIG. 1 is a schematic side view of an apparatus for the manufacturing the water comprising from about 99.76 to about 99.99% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷, ²H₂¹⁸O.

FIG. 2 shows the effect of the light water comprising 99.78% of light isotopologue ¹H₂¹⁶O on succinate-supported H₂O₂ generation by mitochondia as compared to control water comprising 99.73% of light isotopologue ¹H₂¹⁶O.

### DETAILED DESCRIPTION OF THE INVENTION

The present invention provides a substance for treating neoplasm which comprises water comprising from about 99.76 to about 99.99 molecular % of light isotopologue ¹H₂¹⁶O and up to 100% of residual isotopologues.

Further, the present invention provides a method for treating neoplasm which comprises a step of administering to a mammal in need thereof an effective amount of water comprising from about 99.76 to about 99.99 molecular % of light isotopologue ¹H₂¹⁶O and up to 100% of residual isotopologues.

As used herein, the term “isotopologue” is in accordance with IUPAC Compendium of Chemical Terminology 2nd Edition (1997) and refers to a molecular entity that differs only in isotopic composition (number of isotopic substitutions), e.g. ¹H₂¹⁶O, ¹H²H¹⁶O, ¹H₂¹⁸O.

As used herein, the term “residual isotopologues” means ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O. The present invention relates only to water isotopologues comprising stable non-radioactive isotopes of hydrogen and oxygen since the presence of radioactive isotopes is inadmissible for human use.

The water comprising from about 99.76 to about 99.99 molecular % of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O can be prepared by industrial procedures, for example, highly-effective distillation.

The water comprising from about 99.76 to about 99.99 molecular % of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸ may be administered orally, parenterally, topically, or rectally in the method of the invention. Preferably, the effective amount water comprising from about 99.76 to about 99.99 molecular % of light isotopologue ¹H₂¹⁶O and up to 100% of residual isotopologues ¹H₂¹⁷O, ¹H₂¹⁸O, ¹H²H¹⁶O, ¹H²H¹⁷O ¹H²H¹⁸O, ²H₂¹⁶O, ²H₂¹⁷O, ²H₂¹⁸O is administered orally or parenterally.

The water comprising from about 99.76 to about 99.99 molecular % of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O may be administered for 1 day or longer in the method of the invention. Preferably, the effective amount water comprising from about 99.76 to about 99.99 molecular % of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O is administered for all treating period up to achieving a complete remission.

Preferably, the effective amount of the water comprising from about 99.76 to about 99.99 molecular % of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O is 0.5 to 50 ml per day per kg body weight of mammals. By volume, the effective amount of the water comprising from about 99.76 to about 99.99 molecular % of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O is 0.5 to 3000 ml per day per mammal.

Preferably, the method of the invention further comprises a step of administering to a mammal in need thereof from 0.1 to 50 mg per kg body weight of succinic acid or a pharmaceutically acceptable salt thereof. Succinic acid or salts thereof can be administered by steps or simultaneously with water comprising from about 99.76 to about 99.99 molecular % of light isotopologue ¹H₂¹⁶O and up to 100% of residual isotopologues ¹H₂¹⁷O, ¹H₂¹⁸O, ¹H²H¹⁶O, ¹H²_{H}¹⁷O, ¹H²H¹⁸O, ²H₂¹⁶O, ²H₂¹⁷O, ²H₂¹⁸O. The simultaneous method is preferred.

Succinic acid has the chemical structure given below:

HOOCCH₂CH₂COOH

The pharmaceutically acceptable salt of the succinic acid is prepared by known methods from organic and inorganic bases. Such bases include, but are not limited to, nontoxic alkali metal and alkaline earth bases, for example, sodium, potassium, and calcium hydroxide; ammonium hydroxide and nontoxic organic bases, such as triethylamine, butylamine, diethanolamine, and triethanolamine.

The succinic acid or a pharmaceutically acceptable salt thereof may be administered orally, parenterally, topically, or rectally in the method of the invention. Preferably, the effective amount of succinic acid or a pharmaceutically acceptable salt thereof is administered orally or parenterally.

Preferably, the method of the invention further comprises a step of administering to a mammal in need thereof an effective amount of an anti-neoplasm agent. Preferably, the anti-neoplasm agent is selected from the group consisting of mitotic inhibitors, angiogenesis inhibitors, alklylating agents, anti-metabolites, intercalating antibiotics, growth factor inhibitors, cell cycle inhibitors, enzymes, topoisomerase inhibitors, biological response modifiers, anti-hormones, and anti-androgens. Suitable anti-neoplasm agent are all those known by those skilled in the art for the purpose of treating neoplasms. A generally recognized compendium of such anti-neoplasm agents is “The Cancer Chemotherapy Handbook” 6th Edition, Fisher D et al., 2003 or “Lekarstvennye sredstva” 14th Edition, Mashkovsky M, 2002. Anti-neoplasm agent can be administered by steps or simultaneously with water comprising from about 99.76 to about 99.99 molecular % of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸o, ²H₂¹⁶O, ²H₂¹⁷O, ²H₂¹⁸O. The simultaneous method is preferred.

As used herein, the term “anti-neoplasm agent” (used interchangeably with anti-tumor or anti-cancer agent) refers to any agents used in the anti-neoplasm treatment.

Preferably, the method of the invention further comprises a step of radiotherapy of the neoplasm.

Preferably, the method of the invention further comprises a step of surgical treatment of the neoplasm.

As used herein, the term “neoplasm” is in accordance with International Statistical Classification of Diseases and Related Health Problems 10th Revision (ICD-10, Chapter II, Categories C00 to D48) and refers to a disease that is characterized by abnormal growth of new tissue. Preferably, neoplasm to be treated with the method of the present invention is selected from the group consisting of malignant neoplasms, benign neoplasms, in situ neoplasms, and neoplasms of uncertain or unknown behaviour.

The examples of malignant neoplasms include malignant neoplasms of lip, oral cavity and pharynx; digestive organs; respiratory and intrathoracic organs; bone and articular cartilage; skin; mesothelial and soft tissue; breast; female genital organs; male genital organs; urinary tract; eye, brain and other parts of central nervous system; thyroid and other endocrine glands; malignant neoplasms of ill-defined, secondary and unspecified sites; malignant neoplasms, stated or presumed to be primary, of lymphoid, haematopoietic and related tissue; malignant neoplasms of independent (primary) multiple sites.

The examples of benign neoplasms include benign neoplasm of mouth and pharynx; major salivary glands; colon, rectum, anus and anal canal; other and ill-defined parts of digestive system; middle ear and respiratory system; other and unspecified intrathoracic organs; bone and articular cartilage; lipomatous neoplasm; haemangioma and lymphangioma, any site; mesothelial tissue; soft tissue of retroperitoneum and peritoneum; connective and other soft tissue; melanocytic naevi; other benign neoplasms of skin; benign neoplasm of breast; leiomyoma of uterus; other benign neoplasms of uterus; neoplasm of ovary; other and unspecified female genital organs; male genital organs; urinary organs; eye and adnexa; meninges; brain and other parts of central nervous system; thyroid gland; other and unspecified endocrine glands; other and unspecified sites.

The examples of in situ neoplasms include carcinoma in situ of oral cavity, oesophagus and stomach; carcinoma in situ of other and unspecified digestive organs; carcinoma in situ of middle ear and respiratory system; melanoma in situ; carcinoma in situ of skin; carcinoma in situ of breast; carcinoma in situ of cervix uteri; carcinoma in situ of other and unspecified genital organs; carcinoma in situ of other and unspecified sites.

The examples of neoplasms of uncertain or unknown behaviour include neoplasm of uncertain or unknown behaviour of oral cavity and digestive organs; middle ear and respiratory and intrathoracic organs; female genital organs; male genital organs; urinary organs; meninges; brain and central nervous system; endocrine glands; uncertain or unknown behaviour of other and unspecified sites; other neoplasms of uncertain or unknown behaviour of lymphoid, haematopoietic and related tissue; polycythaemia vera; and myelodysplastic syndromes.

Because of inhibiting proliferation and viability of transformed cells with simultaneous enhancement of proliferation and viability of non-transformed cells, the method of the invention is particularly useful to treat neoplasms without side effects such as bone marrow depression.

Because of safety and non-toxicity of water comprising from about 99.76 to about 99.99 molecular % of light isotopologue ¹H₂¹⁶O, the invention provides particularly advantageous methods for treating neoplasms continuously for a long time and/or between courses of conventional chemotherapy, radiotherapy, or surgical treatment to avoid recurrent neoplasms.

In the method of the invention, the compounds of the invention may be formulated in a variety of unit dosage forms well-known from the art. Such forms include, but are not limited to, solutions, solutions for drinking, solutions for injections, solutions for sprays or aerosols, gel tablets, topical solutions, and topical gels or creams.

Herein and after, the term “light water” refers to water comprising from about 99.76% to about 99.99 molecular % of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O.

Herein and after, the content of isotopologues is expressed in molecular %.

The following examples are presented to demonstrate the invention. The examples are illustrative only and are not intended to limit the scope of the invention in any way.

### EXAMPLE 1

This example illustrates the method for producing light water of the invention.

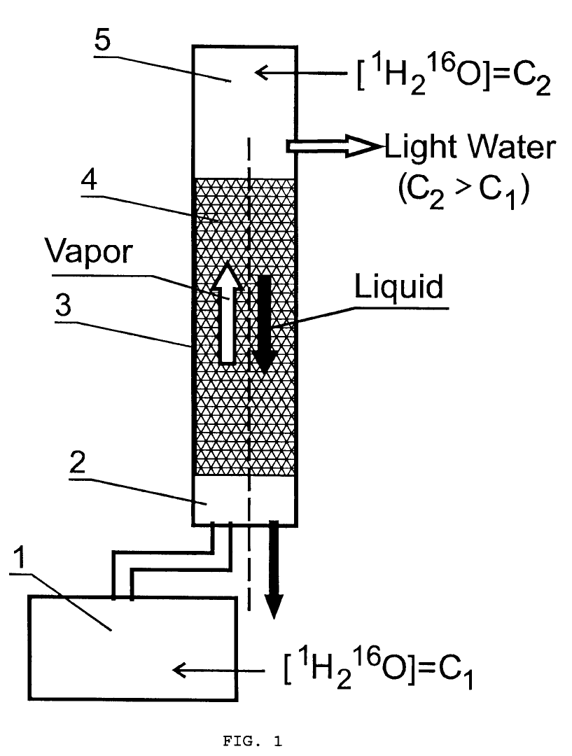

Light water comprising 99.76 to 99.99% of light isotopologue ¹H₂¹⁶O and up to 100% of residual isotopologues (¹H₂¹⁷O, ¹H₂¹⁸O, ¹H²H¹⁶O, ¹H²H¹⁷O, ¹H²H¹⁸o, ²H₂¹⁶O, ²H₂¹⁷O, ²H₂¹⁸O) is prepared by distillation of natural water comprising 99.73% of light isotopologue ¹H₂¹⁶O and up to 100% of residual isotopologues (¹H₂¹⁷O, ¹H₂¹⁸O, ¹H²H¹⁶O, ¹H²H¹⁷O, ¹H²H¹⁸O, ²H₂¹⁶O, ²H₂¹⁷O, ²H₂¹⁸O) with using the apparatus of FIG. 1. The process of the distillation comprises evaporating natural water comprising 99.73% (C₁) of light isotopologue ¹H₂¹⁶O in boiling means 1; supplying the water vapor to the bottom 2 of distillation column 3; contacting between a descending liquid and an ascending vapor mainly on the surface of the contact device 4 (e.g. structured or random packing) within the distillation column; condensing water vapor with concentration of light isotopologue ¹H₂¹⁶O 99.76 to 99.99% (C₂) on condenser 5 installed on upper bound of the distillation column 3; and collecting a part of condensate as condensed light water comprising from 99.76 to 99.99% of light isotopologue ¹H₂ ¹⁶O (C₂>C₁). Resulted light water is used in examples of the invention.

### EXAMPLE 2

This example illustrates isotope effect of light water on succinate-supported H₂O₂ generation in mitochondria.

Materials. Control water comprising 99.73% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸o, ²H₂¹⁶O, ²H₂¹⁷O, ²H₂¹⁸O was used. Light water comprising 99.78% of light isotopologue ¹H₂¹⁶O and up to 100% of residual isotopologues ¹H₂¹⁷O, ¹H₂¹⁸O, ¹H₂H¹⁶O, ¹H²H¹⁷O, ¹H²H^{l8}O, ²H₂¹⁶O, ²H₂¹⁷O, ²H₂¹⁸O was used.

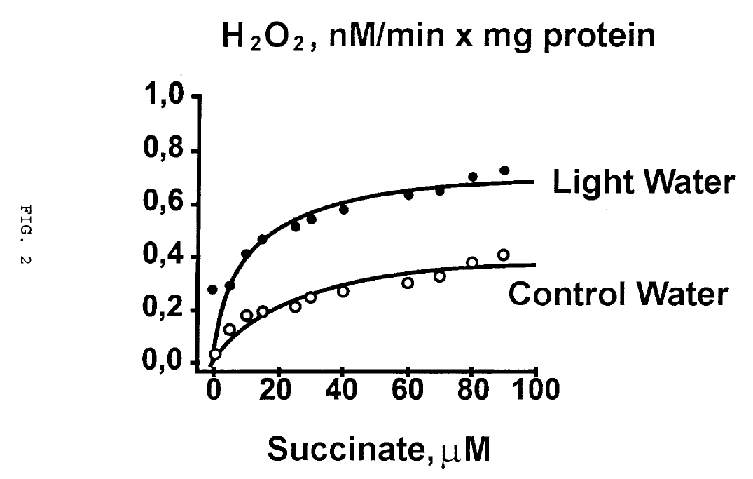

Procedure. Rat liver mitochondria were exposed to different concentrations of disodium succinate (Succinate) solved on the control water or light water. Rates of hydrogen peroxide generation were measured. Data are presented in Table 1 and FIG. 2 as rate of H₂O₂ generation by mitochondria mean ±SD (n=4).

Table 1 shows that light water provides 7-fold increase in the rate of H₂O₂ production by mitochondria as compared to control water at endogenous levels of succinate concentration (exogenous succinate 0 μM).

Table 1 shows that light water provides more than 2-fold increase in the rate of H₂O₂ production by mitochondria as compared to control water at exogenous succinate concentrations from 5 to 90 μM. As shown, absolute rates of H₂O₂ production significantly rise with elevation of succinate concentrations, especially in light water.

The rate of H₂O₂ production by mitochondria on succinate concentrations submits satisfactory to Michaelis-Menten equation V=Vmax[S]/(Km+[S]), wherein Vmax is maximal rate of H₂O₂ production, Km is Michaelis constant, and [S] is a succinate concentration (see FIG. 2). Solvent isotope effect of light water manifests itself predominantly in 2-fold increase of Vmax (0.71 vs. 0.35 nanomoles per min per mg protein for light water and control water respectively), whereas Km is slightly differs (7.3 vs. 9.5 μM for light water and control water respectively). Thus, heavy water isotopologues inhibit enzymes involved in succinate-supported H₂O₂ generation in predominantly uncompetitive manner. Accordingly, light water de-inhibits these initially inhibited enzymes and, thus, increases the rate of succinate-supported H₂O₂ generation in mitochondria to levels that provide H₂O₂ concentrations sufficient to achieve the significant anti-neoplasm effect.

### EXAMPLE 3

This example illustrates method for treating neoplasms.

Materials. Control water comprising 99.73% of light isotopologue ¹H₂¹⁶O and up to 100% of residual isotopologues ¹H₂¹⁷O, ¹H₂¹⁸O, ¹H₂¹⁶O, ¹H₂H¹⁷O, ¹H²H¹⁸O, ^{2H}₂¹⁶O, ²H₂¹⁷O, ²H₂¹⁸O was used. Light water comprising 99.90% of light isotopologue ¹H₂¹⁶O and up to 100% of residual isotopologues ¹H₂¹⁷O, ¹H₂¹⁸O, ¹H²H¹⁸O, ²H₂¹⁶O, ²H₂¹⁷O, ²H₂¹⁸O was used.

Procedure. DBA/BALB(F1) mice were injected i.p. with 3×10⁶ myeloma NS/0 cells to induces tumor. Myeloma NS/O-bearing mice were treated for 5 days since the third day of tumor implantation singly a day with i.p. injection of 0.5 ml saline made on the control water or 0.5 ml saline made on light water. Tumor masses and percent of mitotic cells in bone marrow were measured on day 21. Data are presented in Table 2 as tumor mass mean ±SD (n=5).

The table 2 shows that light water significantly inhibits tumor mass growth as compared to control water. Simultaneously, light water prevents bone marrow depression and significantly increases percent of mitotic cells in bone marrow up to normal values as compared to control water.

### EXAMPLE 4

This example illustrates method for treating neoplasms.

Materials and tumor implantation procedure were as described in example 3. Interferon-alpha2b, rubomicin, and etoposide were used as typical anti-neoplasm agents.

Myeloma NS/O-bearing mice were treated for 5 days since the third day singly a day with i.p. injection of 0.5 ml saline made on the water (control), 0.5 ml solution of 0.1 mg/mouse disodium succinate hexahydrate (Succinate) made on the control water, 0.5 ml solution of 10⁵ IU/mouse interferon-alpha2b (IFN) made on the control water, 0.5 ml solution of 0.016 mg/mouse rubomicin made on the control water, 0.5 ml solution of 0.03 mg/mouse etoposide made on the control water; 0.5 ml saline made on light water, or 0.5 ml solution of 0.1 mg/mouse disodium succinate hexahydrate (Succinate) made on the light water, and 0.5 ml solution of 10⁵ IU/mouse interferon-alpha2b (IFN) made on the light water, 0.5 ml solution of 0.016 mg/mouse rubomicin made on the light water, 0.5 ml solution of 0.03 mg/mouse etoposide made on the light water. Tumor masses were measured on day 21. Data are presented in Table 3 as tumor mass mean ±SD (n=5).

The table 3 shows that combination of light water with succinate significantly inhibits tumor mass growth as compared to control water, control water with succinate, or light water alone. Further, table 3 shows that combination of light water with anti-neoplastic agent significantly inhibits tumor mass growth as compared to control water, control water with anti-neoplastic agent, or light water alone.

### EXAMPLE 5

This example illustrates method for treating neoplasms.

Materials. Waters comprising 99.73 molecular % of light isotopologue ¹H₂¹⁶O (control) and 99.99 molecular % of light isotopologue ¹H₂¹⁶O (experiment) were used.

C57Bl mice were injected i.p. with 3×10⁶ cells EL-4 to induce tumor. Mice were injected for 21 days daily with i.p. injections of 0.5 ml/kg saline made on control water (control) or 0.5 ml/kg saline made on light water. Tumor mass was determined on 22 day. Data are presented in table 4 as tumor mass mean ±SD (n=20).

Table 4 demonstrates that light water significantly inhibits tumor growth as compared to control.

### EXAMPLE 6

This example illustrates method for treating neoplasms.

Materials. Waters comprising 99.73 molecular % of light isotopologue ¹H₂¹⁶O (control) and 99.76 molecular % of light isotopologue ¹H₂¹⁶O were used.

C57Bl mice were injected i.p. with 3×10⁶ cells EL-4 to induce tumor. Mice were allowed to drink for 28 days control or light water daily in amount of 50 ml/kg body weight. Tumor mass was determined on 29 day. Data are presented in table 5 as tumor mass mean ±SD (n=20).

Table 5 demonstrates that light water significantly inhibits tumor growth as compared to control.

### EXAMPLE 7

This example illustrates method for treating neoplasms.

Materials. Waters comprising 99.73 molecular % of light isotopologue ¹H₂¹⁶O (control) and 99.99 molecular % of light isotopologue ¹H₂¹⁶O were used.

C57Bl mice were injected in hip with 1×10⁶ cells ELD to induce solid Erlich's carcinoma. Mice were allowed to drink control or light water daily in amount of 50 ml/kg body weight. Tumor volume was determined on 10 day. Data are presented in table 6 as tumor volume mean ±SD (n=12).

Table 6 demonstrates that light water significantly inhibits tumor growth as compared to control.

### EXAMPLE 8

This example illustrates method for treating neoplasms.

Cells of malignant neoplasms were received from collection of Institute of Cytology, RAN.

Cells were incubated under 37C in 96-well plates for 24 hours in medium made on usual water (control) or light water (99.99% isotopologue ¹H₂¹⁶O, experiment) under succinate concentration of 5 mM. Cell viability was tested by reduction of MTT tetrazolium salt. Apoptosis and cell proliferation. Boehringer Mannheim. 2nd edition. Germany 1998. p. 58. This test determined only living cells, since metabolically active cells reduce MTT to colored formazane while died cells do not. Data are presented in Table 7 as % viable cells mean ±SD (n=10). The percent of viable cells in control is accepted as 100%.

Table 7 demonstrates that light water significantly decreases viability of different type tumor cells both mouse and human as compared to usual water under otherwise equal conditions.

### EXAMPLE 9

This example illustrates method for treating neoplasms.

Man 70 years of age, with benign adenoma of prostate received up to 1.5 L per day of drinking water with light isotopologue ¹H₂¹⁶O content of 99.77% for 4 weeks as a monotherapy. It is found significant decrease in pathological symptomatics linked to the adenoma, including diuresis normalization. Subjectively, some tumor volume decreasing and better feels was observed.

### EXAMPLE 10

The example illustrates method for treating neoplasms.

Woman, 60 years of age, follicular limphoma. She received up to 1.5 L per day of drinking water with light isotopologue ¹H₂¹⁶O content 99.80% in resting period between courses of chemotherapy in the absence of an anti-neoplasm therapy. An increasing duration of a remission time was found from typical 7 months up to 12 months.

Woman, 65 years of age, hairy cell leukemia. She received up to 1.5 L per day of drinking water with light isotopologue ¹H₂¹⁶O content 99.80% in resting period between courses of interferon chemotherapy in the absence of an anti-neoplasm therapy. An increasing duration of a remission time was found from typical 4 months up to 10 months.

Man, 56 years of age, non-Hodgkin's limphoma. He received up to 1.5 L per day of drinking water with light isotopologue ¹H₂¹⁶O content 99.80% in resting period between courses of chemotherapy in the absence of an anti-neoplasm therapy. An increasing duration of a remission time was found from typical 3 months up to 7 months.

TABLE 1
Rate of H₂O₂ Generation

H₂O₂, nanomoles
per min per mg protein

| Succinate, μM | Control water | Light water |
|---|---|---|
| 0 | 0.04 ± 0.01 | 0.27 ± 0.04* |
| 5 | 0.13 ± 0.02 | 0.30 ± 0.02 |
| 10 | 0.18 ± 0.01 | 0.41 ± 0.02* |
| 40 | 0.27 ± 0.01 | 0.58 ± 0.01* |
| 60 | 0.30 ± 0.01 | 0.63 ± 0.01* |
| 70 | 0.34 ± 0.02 | 0.65 ± 0.03* |
| 80 | 0.38 ± 0.01 | 0.70 ± 0.01* |
| 90 | 0.41 ± 0.01 | 0.72 ± 0.02* |

*Denotes statistically significant difference of control (p < 0.05).

TABLE 2

| Treatment | Tumor Mass, mg/mouse |
|---|---|
| Control water | 2230 ± 350 |
| Light water | 1280 ± 140* |

*Denotes statistically significant difference of control (p < 0.05).

TABLE 3

| Treatment | Tumor Mass, mg/mouse |
|---|---|
| Control water | 2230 ± 350 |
| Control water + Succinate | 1780 ± 220* |
| Control water + IFN | 1460 ± 180* |
| Control water + rubomicin | 1270 ± 150* |
| Control water + etoposide | 1050 ± 120* |
| Light water | 1280 ± 140* |
| Light water + Succinate | 310 ± 40* |
| Light water + IFN | 270 ± 50* |
| Light water + rubomicin | 310 ± 40* |
| Light water + etoposide | 280 ± 60* |

*Denotes statistically significant difference of control (p < 0.05).

TABLE 4

| Treatment | Tumor mass, mg/mouse |
|---|---|
| Control | 540 ± 49 |
| Experiment | 432 ± 40* |

*Denotes statistically significant difference of control (p < 0.05).

TABLE 5

| Treatment | Tumor mass, mg/mouse |
|---|---|
| Control | 725 ± 75 |
| Experiment | 490 ± 60* |

*Denotes statistically significant difference of control (p < 0.05).

TABLE 6

| Treatment | Tumor volume, mm³/mouse |
|---|---|
| Control | 795 ± 82 |
| Experiment | 280 ± 61* |

*Denotes statistically significant difference of control (p < 0.05).

TABLE 7

Viable cells, %

| Tumor cells | Experiment | Control |
|---|---|---|
| Murine myeloma NS/0 | 68 ± 7* | 100 ± 11 |
| Human neuroblastoma IMR-32 | 45 ± 6* | 100 ± 13 |
| Human adenocarcinoma HeLa | 72 ± 8* | 100 ± 9 |
| Human epithelial carcinoma A431 | 37 ± 5* | 100 ± 11 |
| Human hepatoma HepG2 | 74 ± 8* | 100 ± 12 |
| Myeloblast leukemia THP-1 | 56 ± 6* | 100 ± 14 |
| T-cell lymphoblastoma Jurkat | 64 ± 9* | 100 ± 10 |
| Glioma T98 | 83 ± 9* | 100 ± 8 |

*Denotes statistically significant difference of control (p < 0.05).

1. A substance for treating neoplasm which comprises water comprising from 99.76 to 99.99 molecular % of light isotopologue ¹H₂¹⁶O and up to 100% of residual isotopologues.

2. A method for treating neoplasm, which comprises a step of administering to a mammal in need thereof an effective amount of water comprising from about 99.76 to about 99.99 molecular % of light isotopologue ¹H₂¹⁶O and up to 100% of residual isotopologues.

3. The method according to claim 2, wherein the effective amount of the water comprising from about 99.76 to about 99.99 molecular % of light isotopologue ¹H₂¹⁶O and up to 100% of residual isotopologues is administered orally or parenterally.

4. The method according to claim 2, further comprising a step of administering to a mammal in need thereof from 0.1 to 50 mg per kg body weight of succinic acid or a pharmaceutically acceptable salt thereof orally or parenterally.

5. The method according to claim 2, wherein the mammal is a human.
